(19) United States
(12) Patent Application Publication
(10) Pub. No.: US 2022/0152426 A1
(43) Pub. Date: May 19, 2022

(54) Low-intensity Focused Ultrasound Treatment Apparatus

(71) Applicant: NEUROSONA Co., Ltd., Seoul (KR)

(72) Inventors: Nam Kuy CHO, Yongin-si (KR); Jin Su KIM, Yongin-si (KR); Min Su YOO, Seoul (KR); Yun Seob YANG, Siheung-si (KR)

(73) Assignee: NEUROSONA Co., Ltd., Seoul (KR)

(21) Appl. No.: 17/117,123

(22) Filed: Dec. 10, 2020

(30) Foreign Application Priority Data
Nov. 18, 2020 (KR) 10-2020-0145064

(51) Int. Cl. A61N 7/00

(52) U.S. Cl. CPC A61N 7/00; A61N 2007/0078; A61N 2007/006; A61N 2007/0026

(57) ABSTRACT

The present invention relates to a low-intensity focused ultrasound therapeutic device. According to one aspect of the present invention, provided is a low-intensity focused ultrasound therapeutic device which employs a single element ultrasound transducer having a focal region, a focal distance and output intensity that can be provide safer treatment for the deep brain of a patient.

### CROSS-REFERENCE TO RELATED APPLICATION

This application claims priority to Korean Patent Application No. 10-2020-0145064, filed on Nov. 18, 2020, the disclosure of which is incorporated herein by reference in its entirety.

### TECHNICAL FIELD

The present invention relates to a low-intensity focused ultrasound therapeutic device.

### BACKGROUND ART

Ultrasound stimulation therapy is known that can stimulate brain lesions without a physical invasive process. As is well known, among the cranial nerve stimulations, transcranial direct current stimulation or transcranial magnetic stimulation is not suitable for deep brain stimulation or precise treatment.

On the other hand, low-intensity focused ultrasound can safely activate or inhibit the activity of neurons in a targeted area of the deep brain without side effects. By controlling cranial nerves, the low-intensity focused ultrasound is capable of mental disorders such as depressive disorder or schizophrenia, as well as chronic nervous system disease such as Alzheimer's disease or epilepsy. Moreover, the low-intensity focused ultrasound induces a stable cavitation for microbubbles, thereby temporarily opening the cerebrovascular barrier. Accordingly, various therapeutic agents for brain diseases can be delivered to the brain.

However, the conventional low-intensity focused ultrasound therapeutic device has generally employed a so-called “multi-array” transducer in which a plurality of transducers are arranged. For controlling positions of ultrasound focal regions, a beamforming control algorithm allows voltages having different amplitudes or phases to be applied to each transducer element in the array.

However, such a multi-array transducer needs to have not less than 500 or 1,000 of small transducers, thus a relatively large ultrasound unit (including, for example, an ultrasound generator, a linear amplifier, an electric signal generator, a waveform modulator, wirings) is required. Furthermore, complex wrings are also needed depending on a driving signal (e.g. for amplitude or phase control) for each transducer. A complicated circuit for driving is additionally required, as well as complex algorithms for driving control and error control. Accordingly, production costs increase for the low-intensity focused ultrasound therapeutic device.

The multi-array transducer has advantageous effects that the single multi-array transducer can focus on a plurality of regions by phase modulation beamforming, but the effective ultrasound focal region has different positions depending on a targeted treatment site. In other words, since the multi-array transducer has several small transducers arranged in a semicircular structure larger than the brain, in a case of targeting a cortical part of the brain, for example, the upper part of the brain, an angle for incidence of ultrasound on the target is not made by the transducer located at a specific area of the multi-array transducer. Only reduced number of transducers transmit effective ultrasound energy, and thus the ultrasound application is difficult.

Meanwhile, when the low-intensity focused ultrasound beam passes through a patient skull to reach the set focal region, the ultrasound transducer and the patient skill have to be fixed in close contact with each other. In the conventional device, a skull positioning device has substantially fixed the patient skull in a single position in order to secure the ultrasound transducer and the patient skill in close contact with each other Accordingly, the patient cannot move his/her head at all by the skull positioning device during the ultrasound therapy. Therefore, the patient may feel uncomfortable.

### DISCLOSURE OF THE INVENTION

### Problem to be Solved by the Invention

An object of the present invention is to provide a low-intensity focused ultrasound therapeutic device capable of reducing production and maintenance costs.

Another object of the present invention is to provide a low-intensity focused ultrasound therapeutic device without a difference occurring in an effective ultrasound focal region depending on a targeted treatment site.

Still another object of the present invention is to provide a low-intensity focused ultrasound therapeutic device in which an ultrasound unit can be simplified.

Even another object of the present invention is to provide a low-intensity focused ultrasound therapeutic device which employs a single element ultrasound transducer that can be controlled to be precisely positioned for various treatment areas in the deep brain.

Further still another object of the present invention is to provide a low-intensity focused ultrasound therapeutic device capable of adjusting positions and various postures of the single element ultrasound transducer for a patient head.

Further still another object of the present invention is to provide a low-intensity focused ultrasound therapeutic device in which the ultrasound transducer and a patient skull are fixed in close contact each other so that a patient can move his/her head slightly during an ultrasound therapy.

### Means for Solving the Problem

According to one aspect of the present invention, provided is a low-intensity focused ultrasound therapeutic device which employs a single element ultrasound transducer having a focal region, a focal distance and output intensity that can be provide safer treatment for the deep brain of a patient.

The low-intensity focused ultrasound therapeutic device may include a plurality of single element ultrasound transducers having different focal distances, in which the single element ultrasound transducer may be detachably attached so that the single element ultrasound transducer can be replaced by the other single element ultrasound transducer having the desired focal distance, out of those single element ultrasound transducers.

In the low-intensity focused ultrasound therapeutic device, an ultrasound gel layer for adjusting the focal distance may be provided on a bottom surface of the single element ultrasound transducer.

In the low-intensity focused ultrasound therapeutic device, a plurality of strap hooks may be formed on the single element ultrasound transducer, and a plurality of straps which are respectively fixed to the plurality of strap hooks on a head strap attached to a patient skull.

In the low-intensity focused ultrasound therapeutic device, a multi-joint manipulation unit may be detachably coupled to one of the plurality of transducers to precisely adjust a position and a posture of the single element ultrasound transducer.

In the low-intensity focused ultrasound therapeutic device, a free articulating arm may be included in the multi joint manipulation unit to adjust a height and a front-rear position of the single element ultrasound transducer, and a manipulation arm may be included in the multi-joint manipulation unit to be detachably, and rotatably in all directions, coupled to the single element ultrasound transducer.

### Effects of the Invention

According to the present invention, it is possible to provide the low-intensity focused ultrasound therapeutic device capable of reducing production and maintenance costs.

Furthermore, it is possible to provide the low-intensity focused ultrasound therapeutic device without a difference occurring in the effective ultrasound focal region.

It is also possible is to provide the low-intensity focused ultrasound therapeutic device in which the ultrasound unit can be simplified.

It is also possible to provide the low-intensity focused ultrasound therapeutic device which employs the single element ultrasound transducer that can be controlled to be precisely positioned for various treatment areas in the deep brain.

It is also possible to provide the low-intensity focused ultrasound therapeutic device capable of adjusting positions and various postures of the single element ultrasound transducer for the patient head.

It is also possible to provide the low-intensity focused ultrasound therapeutic device in which the ultrasound transducer and the patient skull are fixed in close contact each other so that the patient can move his/her head slightly during the ultrasound therapy.

### BRIEF DESCRIPTION OF DRAWINGS

FIG. 1 is a diagram illustrating output characteristics of a focused ultrasound beam of a single element ultrasound transducer according to one embodiment;

FIG. 2 is a perspective view illustrating a structure in which a multi joint manipulation unit and the single element ultrasound transducer are coupled with each other according to one embodiment;

FIG. 3 is a cross-sectional view illustrating the single element ultrasound transducer according to one embodiment;

FIG. 4 is a perspective view illustrating a low-intensity focused ultrasound therapeutic device according to one embodiment;

FIGS. 5A and 5B are diagrams illustrating a structure for attaching a head strap that fixes the single element ultrasound transducer and a patient head in close contact with each other according to one embodiment; and

FIG. 6 is a diagram illustrating a state of the single element ultrasound transducer as an appearance of a semi-rigid ultrasound gel layer changes according to one embodiment.

### BEST MODE FOR CARRYING OUT THE INVENTION

Hereinafter, the present invention will be described in detail with embodiments referring to the accompanying drawings. It will be appreciated that components of such embodiments may be combined in various way for each embodiment unless otherwise stated or contradictory to each other.

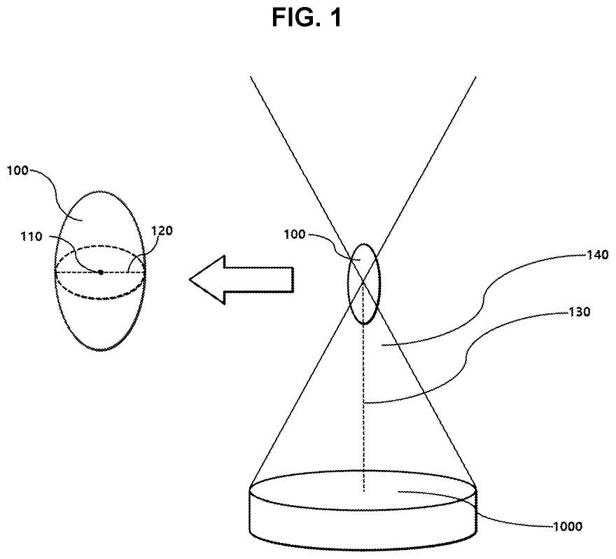

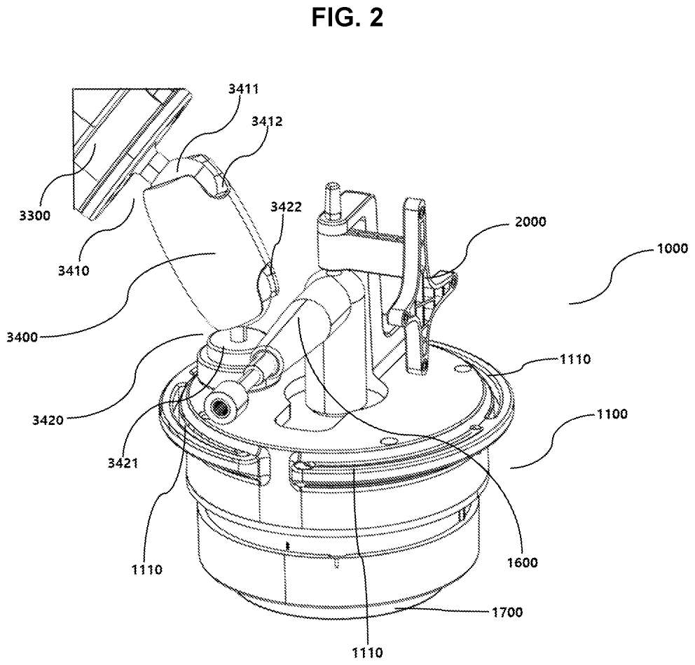

FIG. 1 is a diagram illustrating output characteristics of a focused ultrasound beam of a single element ultrasound transducer according to one embodiment, and FIG. 2 is a perspective view illustrating a structure in which a multi-joint manipulation unit and the single element ultrasound transducer are coupled with each other according to one embodiment. It will be described below with reference to the drawings.

As shown in FIG. 1, the low-intensity focused ultrasound therapeutic device according to the present invention includes a single element ultrasound transducer 1000 that outputs a low-intensity focused ultrasound beam 140, in which a focal region diameter 120 is in a range of 6 mm to 9 mm at a maximum acoustic intensity point 110 within a focal region 100, and a focal length 130 is in a range of 30 mm to 80 mm from a surface thereof to the maximum acoustic intensity point 110; and a transducer marker 2000 that is fixed to the single element ultrasound transducer 1000 to provide information on a position and a pasture of the single element ultrasound transducer 1000.

According to the present invention, the single element ultrasound transducer 1000 is employed in the low-intensity focused ultrasound therapeutic device. The single element ultrasound transducer 1000 has the focal region 100, the focal region diameter 120, the focal distance 130 and the low output intensity, which enables the safe treatment of the patient's deep brain.

According to the present invention, the focal region diameter 120 is in a range of 6 mm to 9 mm at the maximum acoustic intensity point 110 within the focal region 100, in which the energy of the ultrasound beam output from the single ultrasound transducer 1000. The focal distance 130 from the surface of the single element ultrasound transducer 1000 to the maximum acoustic intensity point 110 is in a range of 30 mm to 80 mm. The acoustic intensity of the ultrasound beam output from the single element ultrasound transducer 1000 is low enough to enable the safe treatment for the patient's deep brain. In one embodiment, an acoustic intensity of the ultrasound beam output by the single element ultrasound transducer 1000 at the focal region 100 is 50 W/cm2 or less.

As shown in FIG. 2, according to the present invention, the low-intensity focused ultrasound therapeutic device includes the transducer marker 2000 that is fixed to the single element ultrasound transducer 1000 to provide the information on the position and the pasture of the single element ultrasound transducer 1000.

The position and posture information of the single element ultrasound transducer 1000 is provided by the transducer marker 2000. A position tracker (not shown) included in the low-intensity focused ultrasound therapeutic device can recognize the position and the posture of the single element ultrasound transducer 1000 in a space, provided by the transducer marker 2000, and displays the position and the posture on a monitor (not shown) included in the low-intensity focused ultrasound therapeutic device. Accordingly, for example, a user may move the single element ultrasound transducer 1000 to match the focal region 100 of the transducer with a treatment area inside the patient brain.

Registration can be carried out using a transducer marker (such as reflector or coil), headgear/strap maker (such as reflector or coil), passive tool/pinpoints, or a position tracker (such as optical tracker, IR camera, or electromagnetic tracker) so that a physical space of the actual patient matches with a space on a three-dimensional image captured by CT or MRI. The single element ultrasound transducer 1000 is then moved to match the focal region 100 of the transducer with the treatment area inside the patient brain. More detailed description will be omitted since the registration in which the image space matches with the actual space is already well known.

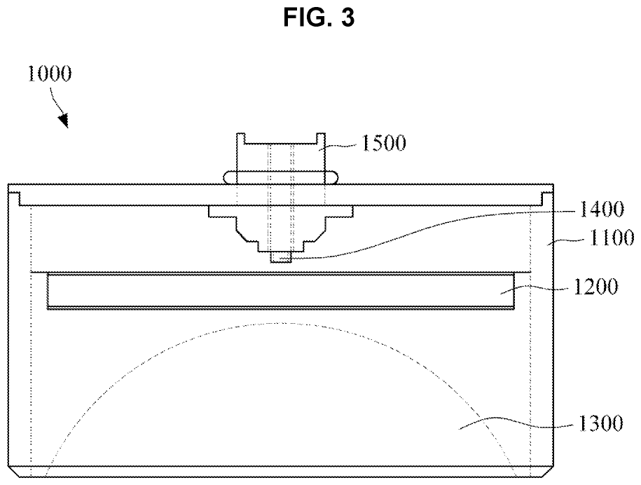

FIG. 3 is a cross-sectional view illustrating the single element ultrasound transducer according to one embodiment. It will be described hereinbelow referring to FIG. 3.

As shown in FIG. 3, according to the present invention, the single element ultrasound transducer 1000 may include an electrode 1400 that applies a voltage; a single piezoelectric element 1200 that outputs a low-intensity ultrasound beam; a spherical lens 1300 that focuses the low-intensity ultrasound beam; a connector 1500 electrically connecting an external control cable (not shown) and the electrode 1400; and a transducer housing 1100 that arranges the electrode 1400, the single piezoelectric element 1200, the spherical lens 1300 and the connector 1500.

In one embodiment, the single piezoelectric element 1200 that outputs the low-intensity ultrasound beam, and the spherical lens 1300 that focuses the low-intensity ultrasound beam on a lower portion of an ultrasound beam output surface of the single piezoelectric element 1200 are arranged in the transducer housing 1100. The output characteristics of the ultrasound beam output by the single piezoelectric element 1200 and the spherical lens 1300 are as described in FIG. 1.

In another embodiment, the single element ultrasound transducer 1000 may further includes a connector cover 1600 that protects the connector 1500 (See FIG. 2). The connector cover 1600 protects a portion of the connector 1500 exposed to the outside of the transducer housing 1100. As shown in FIG. 2, the connector cover 1600 is substantially “L”-shaped and may have a through hole therein. The external control cable (not shown) may be electrically connected to the connector 1500 via the through hole.

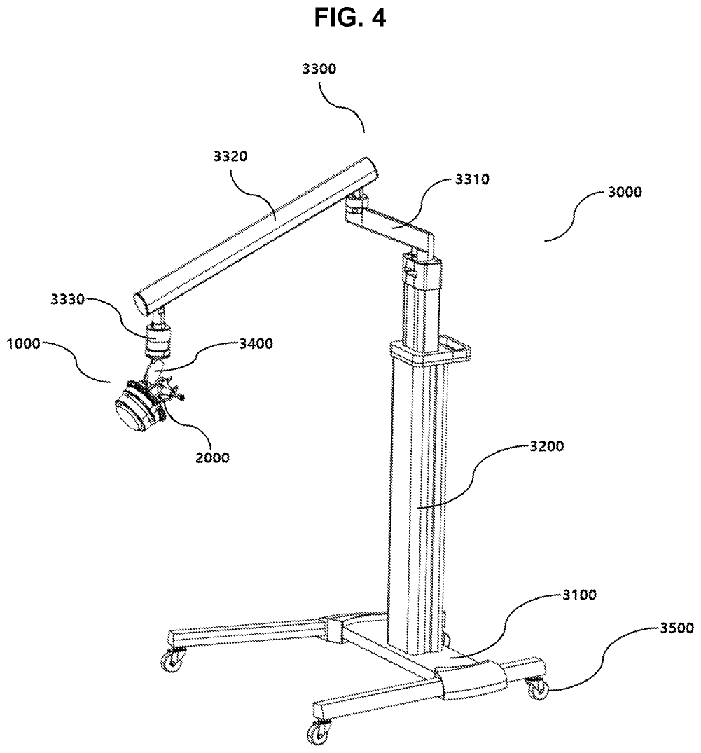

FIG. 4 is a perspective view illustrating a low-intensity focused ultrasound therapeutic device according to one embodiment. It will be described hereinbelow referring to FIG. 4.

As shown in FIG. 4, according to the present invention, the low-intensity focused ultrasound therapeutic device may further include a multi-joint manipulation unit 3000 coupled to the single element ultrasound transducer 1000 so as to adjust the position and the posture of the single element ultrasound transducer.

In one embodiment, the single element ultrasound transducer 1000 may be coupled to the multi-joint manipulation unit 3000. The multi-joint manipulation unit 3000 includes a plurality of joints, and a health care professional may control the plurality of joints coupled to the single element ultrasound transducer 1000. Accordingly, the position and the posture of the single element ultrasound transducer 1000 may be variously adjusted with respect to the patient head.

In one embodiment, the multi joint manipulation unit (3000) and the single element transducer (1000) may be fixedly coupled to each other. Alternatively, the single element ultrasound transducer 1000 may be rotatably coupled to the multi joint manipulation unit 3000. When rotatably coupled, the health care professional can further variously adjust the position and the posture of the single element ultrasound transducer 1000 with respect to the patient head.

According to the present invention, the low-intensity focused ultrasound therapeutic device may further include a plurality of single element ultrasound transducers 1000 having different focal distances; and a detachable unit 3400 (see FIG. 2) that detachably couples any one of the plurality of single element ultrasound transducers 1000 to the multi joint manipulation unit 3000.

As is well known, the single element ultrasound transducer 1000 is limited in varying the focal distance 130, different from the conventional multi-array ultrasound transducer. In one embodiment, the plurality of single element ultrasound transducers 1000 may be required for each focal distance in order to correspond to various focal distances 130. In one embodiment, three single element ultrasound transducers 1000 may be employed in the low-intensity focused ultrasound treatment apparatus for the focal distance of 37 mm, 70 mm, and 80 mm, respectively.

In one embodiment, the detachable unit 3400 may detachably couple any one of the plurality of single element ultrasound transducers 1000 to the multi-joint manipulation unit 3000. In one embodiment, the detachable unit 3400 may include a cylindrical portion (not shown) disposed on an upper surface of the housing 1100 of the single element ultrasound transducer 1000 and having a spiral groove formed on an outer peripheral surface thereof, and a lid portion (not shown) disposed on a coupling surface of the multi joint manipulation unit 3000 and having a spiral protrusion formed on an inner surface thereof, whereby the spiral protrusion is spirally coupled to the spiral groove formed on the outer peripheral surface of the cylindrical portion. Accordingly, for example, the single element ultrasound transducer 1000 for a 37-mm focal distance or the single element ultrasound transducer 1000 for a 70-mm focal distance may be selectively coupled to the multi joint manipulation unit 3000.

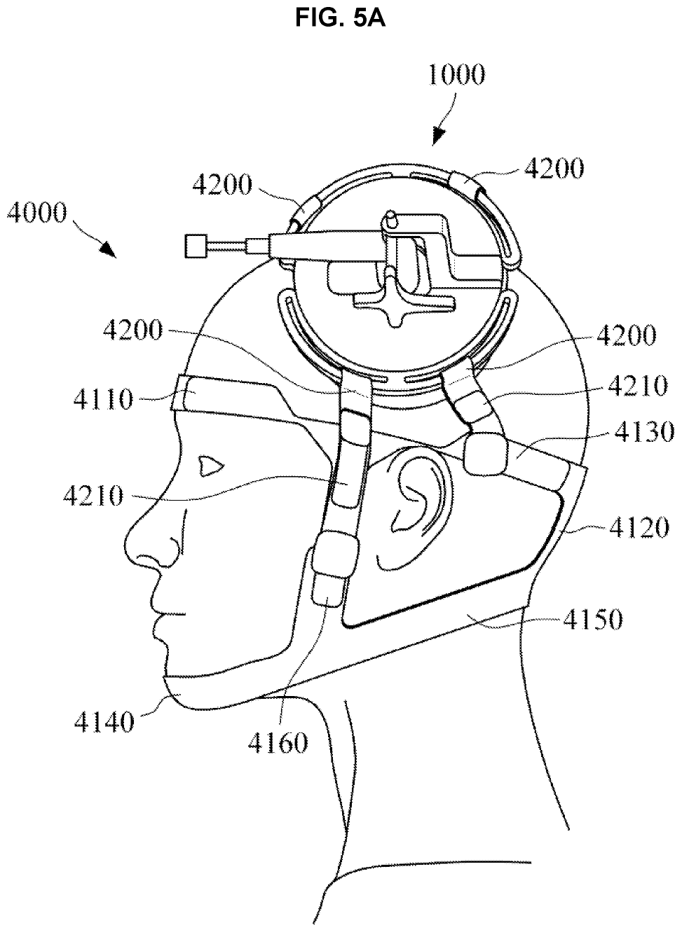

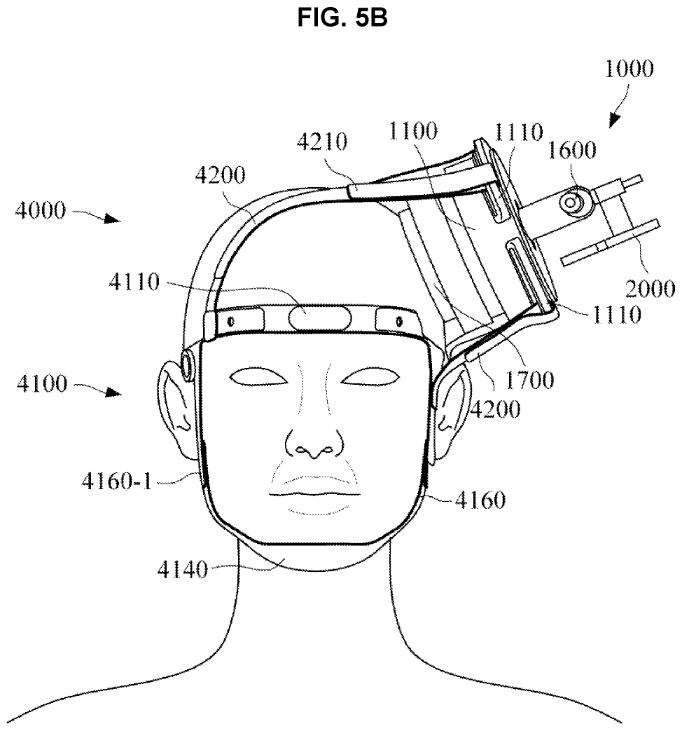

FIGS. 5A and 5B are diagrams illustrating a structure for attaching a head strap that fixes the single element ultrasound transducer and a patient head in close contact with each other according to one embodiment. It will be described hereinbelow referring to FIG. 5.

As shown in FIGS. 5A and 5B, according to the present invention, the low-intensity focused ultrasound therapeutic device may further include a plurality of strap hooks 1110 formed along an edge of the single element ultrasound transducer 1000; and a head strap 4000 including a plurality of straps which are fixed while wrapping up a patient head between the plurality of strap hooks 1110.

According to the present invention, the plurality of strap hooks 1110 may be formed along an edge of an upper surface of the transducer housing 1100. In one embodiment, four grooves 1110 on which a strap can be hooked may be formed on the upper surface of the transducer housing 1100. In one embodiment, the head strap 4000 may include a head strap main body 4100 mounted on the patient head, and a plurality of fixing straps 4200 fixed to the plurality of strap hooks 1110. In one embodiment, when the patient wears the head strap 4000 on his/her head, the head strap main body 4100 may closely support a plurality of portions on the patient head. In one embodiment, the plurality of straps supporting the plurality of head portions may be integrally formed.

In one embodiment, the head strap main body 4100 may include a frontal region strap 4110 supporting a frontal region of the head; a mandible lower jaw strap 4140 supporting a mandible lower jaw of the head; a regio occipitalis strap 4120 supporting a regio occipitalis of the head; an upper left connecting strap 4130 connecting the frontal region strap 4110 and the regio occipitalis strap 4120; an upper right connecting strap 4130-1 connecting the frontal region strap 4110 and the regio occipitalis strap 4120; a lower left connecting strap 4150 connecting the mandible lower jaw strap 4140 and the regio occipitalis strap 4150; a lower right connecting strap 4150-1 connecting the mandible lower jaw strap 4140 and the regio occipitalis strap 4120; a mandible left strap 4160 vertically connecting the upper left connecting strap 4130 and the lower left connecting strap 4150, and a mandible right strap 4160-1 vertically connecting the upper right connecting strap 4130-1 and the lower right connecting strap 4150-1. Furthermore, the head strap main body 4100 may support the plurality of portions on the patient head in various ways. In one embodiment, the head strap main body 4100 may be manufactured so that a strength of contact with the patient head can be adjusted.

According to the present invention, the plurality of fixing straps 4200 may be fixed to the plurality of strap hooks 1110. In one embodiment, one end of each fixing strap 4200 may be fixed to a corresponding portion of the head strap main body 4100. The other end of each fixing strap 4200 may be inserted into a groove of each strap 1110. The other end of each fixing strap 4200 may be fixed by a fixing surface 4210 formed on the plurality of fixing straps 4200 or the head strap main body 4100. In one embodiment, a Velcro male portion may be formed at the other end of each fixing strap 4200 while a Velcro female portion may be formed on the fixing surface 4210.

According to the present invention, it is possible to fix between the patient head and the single element ultrasound transducer 1000 using the head strap 4000. By using the head strap 4000 to closely fix the single element ultrasound transducer 1000, the patient can move his/her head slightly during the ultrasound therapy. Furthermore, since the single element ultrasound transducer 1000 is mounted on the multi joint manipulation unit 3000, the patient can comfortably receive the treatment without feeling uncomfortable due to the weight and size of the single element ultrasound transducer 1000.

In one embodiment, the plurality of fixing straps 4200 may be made of non-elastic material. Consequently, the single element ultrasound transducer 1000 can be fixed without movement.

According to the present invention, as shown in FIGS. 2 and 4, the multi joint manipulation unit 3000 may include a support 3100; a stand 3200 vertically extending from the support 3100; and free articulating arm 3300 of which one end is coupled to an upper portion of the stand 3200 and which is bendable around at least one joint axis.

The support 3100 may support components included in the low-intensity focused ultrasound therapeutic device. The support 3100 may support the stand 3200, the free articulating arm 3300, the detachable unit 3400, and the single element ultrasound transducer 1000 coupled to the detachable unit 3400. A plurality of wheels 3500 are disposed on a bottom surface of the support 3100 so that the low-intensity focused ultrasound therapeutic device can be moved.

The stand 3200 may extend vertically from the support 3100 and may have a certain height. One end of the free articulating arm 3300 may be coupled to the upper portion of the stand 3200. The free articulating arm 3300 may be bendable around at least one joint axis. In one embodiment, the joint axis may be a rotation shaft. For example, two arms are rotatably connected to any rotation shaft disposed on the free articulating arm 3300, and the two arms may be bent at a predetermined angle with respect to the rotation shaft. The two arms can be rotated little by little with respect to the rotation shaft as manipulated by the user.

As shown in FIG. 2, according to the present invention, the detachable unit 3400 has one end that is rotatably coupled to the other end of the free articulating arm 3300 and the other end that is rotatably coupled to the single element ultrasound transducer 1000. Consequently, the health care professional can further variously adjust the position and the posture of the single element ultrasound transducer 1000 with respect to the patient head by manipulating the detachable unit 3400. In one embodiment, the one end of the detachable unit 3400 and the other end of the free articulating arm 3300 may be rotatably coupled with respect to a first rotation shaft (not shown) disposed on the detachable unit 3400. In one embodiment, the other end of the detachable unit 3400 and the single element ultrasound transducer 1000 may be rotatably coupled with respect to a second rotation shaft (not shown) disposed on the detachable unit 3400.

As shown in FIG. 2, according to the present invention, the detachable unit 3400 may include a first ball stud 3411 fixed to the other end of the free articulating arm 3300; a first socket space 3412 surrounding a ball included in the first ball stud 3411 and rotating in all directions with respect to a surface of the ball; a second ball stud 3421 fixed to the single element ultrasound transducer 1000; and a second socket space 3422 surrounding a ball included in the second ball stud 3421 and rotating in all directions with respect to a surface of the ball.

A first plate included in the first ball stud 3411 of the detachable unit 3400 may be fixed to the other end of the free articulating arm 3300. A first ball may be formed at an end of the first stud extending from the first plate. The first socket space 3412 included in the detachable unit 3400 may surround the first ball included in the first ball stud 3411 and rotate in all directions with respect to a surface of the first ball.

A second plate included in the second ball stud 3421 of the detachable unit 3400 may be fixed to the upper portion of the transducer housing 1100. A second ball may be formed at an end of the second stud extending from the second plate. The second socket space 3422 included in the detachable unit 3400 may surround the second ball included in the second ball stud 3421 and rotate in all directions with respect to a surface of the second ball.

Consequently, the health care professional can further variously adjust the position and the posture of the single element ultrasound transducer 1000 with respect to the patient head by manipulating the detachable unit 3400. Since the socket spaces 3412 and 3422 can be separated from the ball studs 3411 and 3421, respectively, the single element ultrasound transducer 1000 having a specific focal distance can be selectively mounted on the low-intensity focused ultrasound therapeutic device.

According to the present invention, as shown in FIG. 4, the free articulating arm 3300 may include a first arm 3310 of which one end is rotatably coupled to the upper portion of the stand 3200 with respect to a first vertical rotation shaft; a second arm 3320 of which one end is coupled to the other end of the first arm 3310 rotatably with respect to a second vertical rotation shaft and rotatably with respect to a first horizontal rotation shaft; and a third arm 3330 of which one end is rotatably coupled to the other end of the second arm 3320 with respect to a second horizontal rotation shaft.

In one embodiment, the one end of the first arm 3310 may be rotatably coupled to the first vertical rotation shaft (not shown) disposed inside the stand 3200 and rotate left and right with respect to the first vertical rotation shaft as manipulated by the health care professional. In one embodiment, the first arm 3310 may extend in an upward direction with an obtuse angle falling in a range of 90 degrees to 180 degrees with respect to the vertical stand 3200.

According to the present invention, the one end of the second arm 3320 may be rotatably coupled to the other end of the first arm 3310 with respect to the second vertical rotation shaft. In one embodiment, the one end of the second arm 3320 may be rotatably coupled to the second vertical rotation shaft disposed inside the other end of the first arm 3310. Consequently, the second arm 3320 may rotate left and right with respect to the second vertical rotation shaft as manipulated by the health care professional. Additionally, the one end of the second arm 3320 may be rotatably coupled to the other end of the first arm 3310 with respect to the first horizontal rotation shaft (not shown). In one embodiment, the first horizontal rotation shaft may be formed along a through hole (not shown) formed in a horizontal direction in the second vertical rotation shaft disposed inside the other end of the first arm 3310. That is, the second horizontal rotation shaft (not shown) may be inserted into the through hole in the horizontal direction. Both ends of the second horizontal rotation shaft may be fixed to an inner wall of the second arm 3320. Consequently, the second arm 3320 may rotate in up and down directions with respect to the first horizontal rotation shaft as manipulated by the health care professional. In one embodiment, the second arm 3320 may extend in a downward direction with an acute angle falling in a range of 1 degree to 90 degrees with respect to the second vertical rotation shaft.

Accordingly, the first arm 3310 and the second arm 3320 can be rotated left and right around the second vertical rotation shaft, and can rotate up and down around the first horizontal rotation shaft. In a preferred embodiment, the health care professional may adjust a height of the single element ultrasound transducer 1000 by rotating the second arm 3320 in the vertical direction. Moreover, the health care professional may adjust the position of the single element ultrasound transducer 1000 in a backward direction (toward the stand 3200) by, for example, rotating the first arm 3310 to the right with respect to the first vertical rotation shaft and rotating the second arm 3320 to the left with respect to the second vertical rotation shaft. On the contrary, the health care professional may adjust the position of the single element ultrasound transducer 1000 in a forward direction (moving away from the stand 3200) by rotating the first arm 3310 and the second arm 3320 so that the two arms 3310 and 3320 are aligned when viewed from above.

In one embodiment, the one end of the third arm 3330 may be rotatably coupled to the other end of the second arm 3320 with respect to the second horizontal rotation shaft (not shown). The one end of the third arm 3330 may be rotatably coupled to the second vertical rotation shaft disposed inside the other end of the second arm 3320. In one embodiment, when the health care professional moves the second arm 3320 upward or downward, the third arm 3330 may rotate in a first direction (for example, in a direction toward the stand 3200) or in a second direction (for example, in a direction moving away from the stand 3200) with respect to the second horizontal rotation shaft. Accordingly, the third arm 3330 can always be maintained the vertical direction.

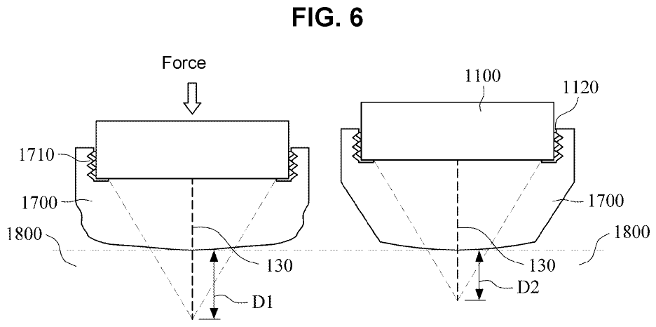

FIG. 6 is a diagram illustrating a state of the single element ultrasound transducer as an appearance of a semi-rigid ultrasound gel layer changes according to one embodiment. It will be described hereinbelow referring to FIGS. 2 and 6.

As shown in FIG. 6, the low-intensity focused ultrasound device may further include an ultrasound gel layer 1700 that is detachably disposed on a bottom surface of the single element ultrasound transducer 1000 and through which the ultrasound beam is transmitted in a space between a patient skin 1800 and the single element ultrasound transducer 1000.

As is well known, if there is air between the patient skin 1800 (e.g. scalp) and the single element ultrasound transducer 1000, the ultrasound beam is not properly transmitted to the internal organs of the patient due to, for example, attenuation. Accordingly, the ultrasound gel layer 1700 prevents a phenomenon in which the focused ultrasound beam is not transmitted due to a space that may be formed between the single element ultrasound transducer 1000 and the patient skin. The ultrasound gel layer 1700 is formed of an ultrasound propagation medium (couplant) and serves to fill such a space stated above.

As already known, the ultrasound gel layer 1700 may be a semi-rigid medium (see KR10-1585301B) or a solid medium (see to KB2012-0118864A).

The ultrasound gel layer 1700, as disclosed in KB2012-0118864A, may be a solid but stretchable/elastic lid (alternatively, a case or a cover may be employed), of which an inner space is empty so that it can be fitted to an outer peripheral surface of a lower portion of the transducer housing 1100 of the single element ultrasound transducer 1000. Accordingly, the ultrasound gel layer 1700 may be detachably disposed on the bottom surface of the single element ultrasound transducer 1000.

Alternatively, as disclosed in KR10-1585301B, the transducer housing 1100 of the single element ultrasound transducer 1000 may have a cylindrical shape (see FIG. 2) when the single piezoelectric element 1200 provided therein has a circular shape. The spiral protrusion (see reference number 1120 of FIG. 6) may be formed on an inner peripheral surface of the transducer housing 1100. The ultrasound gel layer 1700 may have a shape of the lid (may be the case or the cover) that is empty inside thereof, and the spiral groove (see reference number 1710 of FIG. 6) may be formed on an inner peripheral surface of the lid. Accordingly, the ultrasound gel layer 1700 may be detachably using a spiral coupling on the bottom surface of the single element ultrasound transducer 1000. Accordingly, the specific ultrasound gel layer 1700 corresponding to the certain single element ultrasound transducer 1000 having the specific focal distance may be selected and detachably disposed on the bottom surface of the single element ultrasound transducer 1000.

As shown in FIG. 6, according to the present invention, the ultrasound gel layer 1700 is a semi-rigid medium of which appearance is deformed depending on a magnitude and a direction of a force F applied on the single element ultrasound transducer 1000 due to an appearance of the patient skin 1800 and user's manipulation, and a distance D2 between a surface of the patient skin 1800 and a target treatment area within the patient skin is varied (D1 to D2) as the appearance of the ultrasound gel layer is changed.

In one embodiment, the ultrasound gel layer 1700 is a semi-rigid medium and has flexibility, elasticity and compressibility, whereby its appearance may be deformed according to the appearance of the patient skin 1800. In one embodiment, the ultrasound gel layer 1700 may be a semi-rigid hydrogel. As disclosed in KR10-1585301B, hydrophilic polyvinyl alcohol (PVA) without irritation to the human body may be adopted as the semi-rigid hydrogel.

In one embodiment, the appearance of the semi-rigid ultrasound gel layer 1700 may be changed in response to the magnitude and the direction of the force F applied to the single element ultrasound transducer 1000 by the user's manipulation. As the appearance is modified, the ultrasound gel layer 1700 may vary (D2 to D1) the distance D between the surface of the patient skin 1800 and the target treatment area within the patient skin.

As shown in FIG. 6, the focal distance 130 is never changed. Before the semi-rigid ultrasound gel layer 1700 is deformed by, for example, compression, the distance between the surface of the patient skin 1800 and the target treatment area within the patient skin is represented by D2. However, when the semi-rigid ultrasound gel layer 1700 is deformed by, for example, compression, the distance between the surface of the patient skin 1800 and the target treatment area within the patient skin is varied to Dl. That is, while the focal distance 130 is not changed, the single element ultrasound transducer 1000 can treat the target treatment area which is deeper inside the surface of the patient skin 1800 owning to the semi-rigid ultrasound gel layer 1700. In other words, the single element ultrasound transducer 1000 can ensure more precise treatment for target treatment areas at various locations inside the patient skin.

The present invention has been described through embodiments with reference to the accompanying drawings, but the present invention is not limited thereto; and it should be interpreted that the present invention encompasses various improvements and modifications that will be appreciated to those skilled in the art. Those improvements and modifications are fully encompassed in the scope of claim.

### DESCRIPTION OF SYMBOLS

1000: single element ultrasound transducer
100: focal region
110: maximum acoustic intensity point
120: focal region diameter
130: focal distance
200: focused ultrasound beam
1100: transducer housing
1110: plurality of band hooks
1120: spiral protrusion
1200: single piezoelectric element
1300: spherical lens
1400: electrode
1500: connector
1600: connector cover
1700: ultrasound gel layer
1710: spiral groove
1800: patient skin
2000: transducer marker
3000: multi joint manipulation unit
3100: support
3200: stand
3300: free articulating arm
3310: first arm
3320: second arm
3330: third arm
3400: detachable unit
3410: first ball joint
3411: first ball stud
3412: first socket space
3420: second ball joint
3421: second ball stud
3422: second socket space
3500: plurality of wheels
4000: head strap
4100: head strap main body
4110: frontal region strap
4120: regio occipitalis strap
4130: upper left connecting strap
4130-1: upper right connecting strap
4140: mandible lower jaw strap
4150: lower left connecting strap
4150-1: lower right connecting strap
4160: mandible left strap
4160-1: mandible right strap
4200: plurality of fixing straps

1. A low-intensity focused ultrasound therapeutic device, comprising:
a single element ultrasound transducer that outputs a low-intensity focused ultrasound beam, wherein a focal region diameter is in a range of 6 mm to 9 mm at a maximum acoustic intensity point within a focal region, and a focal length is in a range of 30 mm to 80 mm from a surface thereof to the maximum acoustic intensity point; and
a transducer marker that is fixed to the single element ultrasound transducer to provide information on a position and a pasture of the single element ultrasound transducer.

2. The low-intensity focused ultrasound therapeutic device according to claim 1, wherein
an acoustic intensity of an ultrasound beam output by the single element ultrasound transducer at the focal region is 50 W/cm2 or less.

3. The low-intensity focused ultrasound therapeutic device according to claim 2, wherein the single element ultrasound transducer includes:
an electrode that applies a voltage;
a single piezoelectric element that outputs a low-intensity ultrasound beam;
a spherical lens that focuses the low-intensity ultrasound beam;
a connector electrically connecting an external control cable and the electrode; and
a transducer housing that arranges the electrode, the single piezoelectric element, the spherical lens and the connector.

4. The low-intensity focused ultrasound therapeutic device according to claim 1, further comprising:
a multi-joint manipulation unit coupled to the single element ultrasound transducer so as to adjust the position and the posture of the single element ultrasound transducer.

5. The low-intensity focused ultrasound therapeutic device according to claim 4, further comprising:
a plurality of single element ultrasound transducers having different focal distances; and
a detachable unit that detachably couples any one of the plurality of single element ultrasound transducers to the multi-joint manipulation unit.

6. The low-intensity focused ultrasound therapeutic device according to claim 4, further comprising:
a plurality of strap hooks formed along an edge of the single element ultrasound transducer; and
a plurality of straps which are fixed while wrapping up a patient head between the plurality of strap hooks.

7. The low-intensity focused ultrasound therapeutic device according to claim 4, wherein the multi-joint manipulation unit includes:
a support;
a stand vertically extending from the support; and
a free articulating arm of which one end is coupled to an upper portion of the stand and which is bendable around at least one joint axis.

8. The low-intensity focused ultrasound therapeutic device according to claim 7, wherein
the detachable unit has one end that is rotatably coupled to the other end of the free articulating arm and the other end that is rotatably coupled to the single element ultrasound transducer.

9. The low-intensity focused ultrasound therapeutic device according to claim 8, wherein the detachable unit includes:
a first ball stud fixed to the other end of the free articulating arm;
a first socket space surrounding a ball included in the first ball stud and rotating in all directions with respect to a surface of the ball;
a second ball stud fixed to the single element ultrasound transducer; and
a second socket space surrounding a ball included in the second ball stud and rotating in all directions with respect to a surface of the ball.

10. The low-intensity focused ultrasound therapeutic device according to claim 7, wherein the free articulating arm includes:
a first arm of which one end is rotatably coupled to the upper portion of the stand with respect to a first vertical rotation shaft;
a second arm of which one end is coupled to the other end of the first arm rotatably with respect to a second vertical rotation shaft and rotatably with respect to a first horizontal rotation shaft; and
a third arm of which one end is rotatably coupled to the other end of the second arm with respect to a second horizontal rotation shaft.

11. The low-intensity focused ultrasound therapeutic device according to claim 5, further comprising:
an ultrasound gel layer that is detachably disposed on a bottom surface of the single element ultrasound transducer and through which the ultrasound beam is transmitted in a space between a patient skin and the single element ultrasound transducer.

12. The low-intensity focused ultrasound therapeutic device according to claim 11, wherein the ultrasound gel layer is a semi-rigid medium of which appearance is deformed depending on a magnitude and a direction of a force applied on the single element ultrasound transducer due to an appearance of the patient skin and user's manipulation, and
a distance between a surface of the patient skin and a target treatment area within the patient skin is varied as the appearance of the ultrasound gel layer is changed.
